(19) United States
(12) Patent Application Publication
(10) Pub. No.: US 2008/0112605 A1
(43) Pub. Date: May 15, 2008

(54) Methods and Apparatus for Automatic Body Part Identification and Localization

(75) Inventors: Lin Hong, Monmouth Junction, NJ (US); Shen Hong, Plainsboro, NJ (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS CORPORATE RESEARCH, INC., PRINCETON, NJ (US)

(21) Appl. No.: 11/933,518

(22) Filed: Nov. 1, 2007

Related U.S. Application Data
(60) Provisional application No. 60/865,692, filed on Nov. 14, 2006.

(51) Int. Cl. A61B 5/00

(52) U.S. Cl. 382/131

(57) ABSTRACT

Methods and apparatus are disclosed for automatically identifying and locating body parts in medical imaging. To automatically identify body parts of in an image, an identification and location algorithm is used. This establishes a reference frame in relation to the image. Then, a location of the head in relation to the frame is established. After upper and lower boundaries of the head are determined, a neck section of the image is identified. The neck section is identified using the lower boundary of the head section. The location of the neck section is then found. A thorax cage section is found and located positively below the neck section. The abdomen and pelvis are identified together and ultimately separately located and identified.

### FIELD OF THE INVENTION

The present invention relates generally to medical imaging and more particularly to automatic body part identification in medical imaging.

### BACKGROUND OF THE INVENTION

In medical imaging, such as computed tomography (CT), computed axial tomography (CAT), magnetic resonance imaging (MRI), and positron emission tomography (PET), it is necessary to identify and locate a target body part for imaging. Conventionally, a full body or a significantly large region that includes the target body part is pre-scanned to produce an overview image (e.g., a two dimensional low resolution x-ray image). A user manually marks the position of the intended target body part on the low resolution overview image to indicate to the medical imaging device the position information of the intended target body part. This manual operation is undesirable in medical image acquisition procedures because it significantly reduces the throughput of the medical image acquisition devices and increases the cost of operation.

Further, intended target body part (e.g., patient) movement may result in acquisition failure. Accordingly, to account for patient movement as well as low image quality, a user may be required to mark a region significantly larger than the intended target body part. This may result in unnecessary exposure of the non-target regions and larger than necessary image data size.

Identifying and locating target body parts reliably from low quality two-dimensional images is difficult. The appearance of body parts in overview images generally exhibits a significant variation across individuals. The position and size of body parts may also vary significantly. Low resolution overview images also tend to be of very low contrast. As such, it may be very difficult to differentiate target body parts and/or organs in these images. In addition, some images may have complex backgrounds which may further obfuscate the target body parts.

Therefore, alternative methods of body part identification and localization are required to improve system throughput and increase reliability in target body part identification and localization in medical imaging.

### BRIEF SUMMARY OF THE INVENTION

The present invention provides methods and apparatus for automatic body part identification and location in medical imaging. To automatically identify and locate body parts of in an image, an identification and location algorithm is used. This method establishes a reference frame in relation to the image. Then, a location of the head in relation to the frame is established. After upper and lower boundaries of the head are determined, a neck section of the image is identified. The neck section is identified using, at least in part, the lower boundary of the head section. The location of the neck section is then determined.

Similarly, a thorax cage section is identified and located positively below the neck section. In turn, the abdomen and pelvis are identified together and ultimately separately located. Various algorithms are employed for determining the boundaries of these body sections.

These and other advantages of the invention will be apparent to those of ordinary skill in the art by reference to the following detailed description and the accompanying drawings.

### BRIEF DESCRIPTION OF THE DRAWINGS

FIG. 1 is a full-body input image;

FIG. 2 is a head section of a full-body input image;

FIG. 3 is an exemplary side-view head section of a full-body input image;

FIG. 4 is a neck section of full-body input image;

FIG. 5 is a thorax cage section of full-body input image;

FIG. 6 is an abdomen and pelvis section of full-body input image;

FIG. 7 is an exemplary side-view abdomen and pelvis section;

FIG. 8 depicts a flowchart of a method 800 of automatic body part identification and localization according to an embodiment of the invention;

FIG. 9 depicts a flowchart of a method 900 of establishing a global reference frame according to a particular embodiment of the present invention;

FIG. 10 depicts a flowchart of a method 1000 of automatically identifying and locating a first body part;

FIG. 11 depicts a flowchart of a method 1100 of automatically identifying and locating a second body part;

FIG. 12 depicts a flowchart of a method 1200 of automatically identifying and locating a third body part;

FIG. 13 depicts a flowchart of a method 1300 of automatically identifying and locating a fourth and/or fifth body part; and

FIG. 14 is a schematic view of a computer according to an embodiment of the present invention.

### DETAILED DESCRIPTION

The present invention generally provides methods and apparatus for automatic body part identification and localization in medical imaging. In at least one embodiment, a body part identification and localization method is performed to identify (e.g., recognize) and locate (e.g., determine the approximate boundaries of) a target body part from an input image. Using the method, commonly examined body parts may be identified and located in various input images. For example, the head section, the neck section, the thorax cage section, the abdomen section, and the pelvis section may be located and identified in both front view images and side view images. The body part identification and localization methods involve estimating global reference properties and applying a sequence of body part algorithms to recognize and locate a body part in an input image relative to the global reference properties.

Though discussed herein as systems and methods for body (e.g., patient, human) part identification and localization, it is understood that the systems and methods may be applied to identifying and/or localizing any whole or portion of any object. Similarly, though discussed herein in relation to medical imaging, the systems and methods may be applied to any imaging application (e.g., facial recognition, surveillance, art authentication, research, etc.).

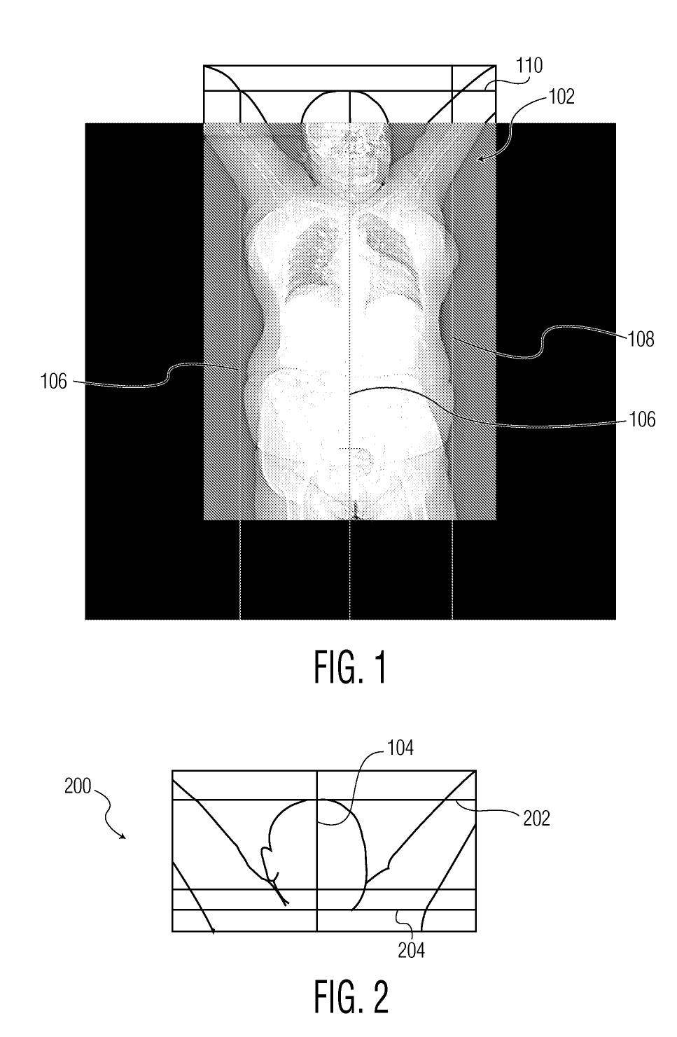

FIGS. 1-7 depict input images that may be used in automatic body part identification and localization. These input images are exemplary only and not intended to show the only possible images which may be used. Such images may come from any appropriate source, such as a CT scan, CAT scan, PET scan, MRI, X-Ray, etc. and may be of any appropriate image quality, resolution, orientation, and/or dimension. For simplicity, the input images discussed below may be overview images that are two dimensional low resolution x-ray images.

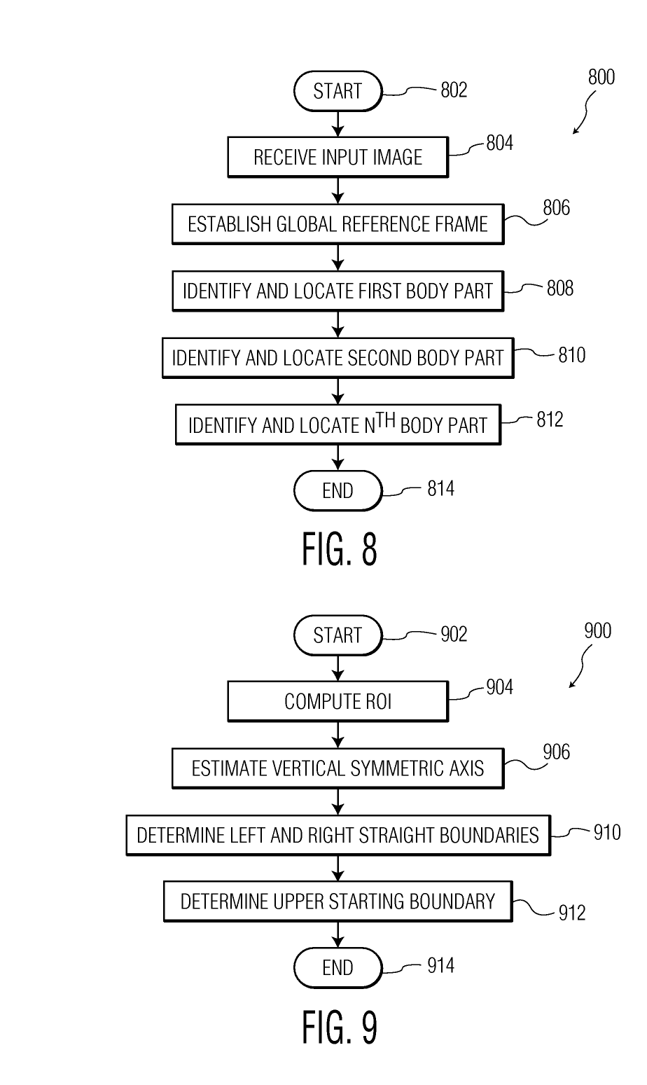

FIG. 1 is a full-body input image 100. Full-body input image 100 may have a number of image properties, discussed in further detail below with respect to methods described below in connection with FIGS. 8 and 9. These image properties may include a region of interest (ROI) 102, a vertical symmetric axis 104, a left straight boundary 106, a right straight boundary 108, and an upper starting boundary 110.

FIG. 2 is a head section 200 of full-body input image 100. Head section 200 is not a separate image, but is the uppermost portion of the full-body input image 100 (e.g., the section likely containing an image of a head of a patient). Head section 200 retains vertical symmetric axis 104. As will be discussed below with respect to methods 800 and 1000, head section 200 may also have a head upper boundary 202 and a head lower boundary 204.

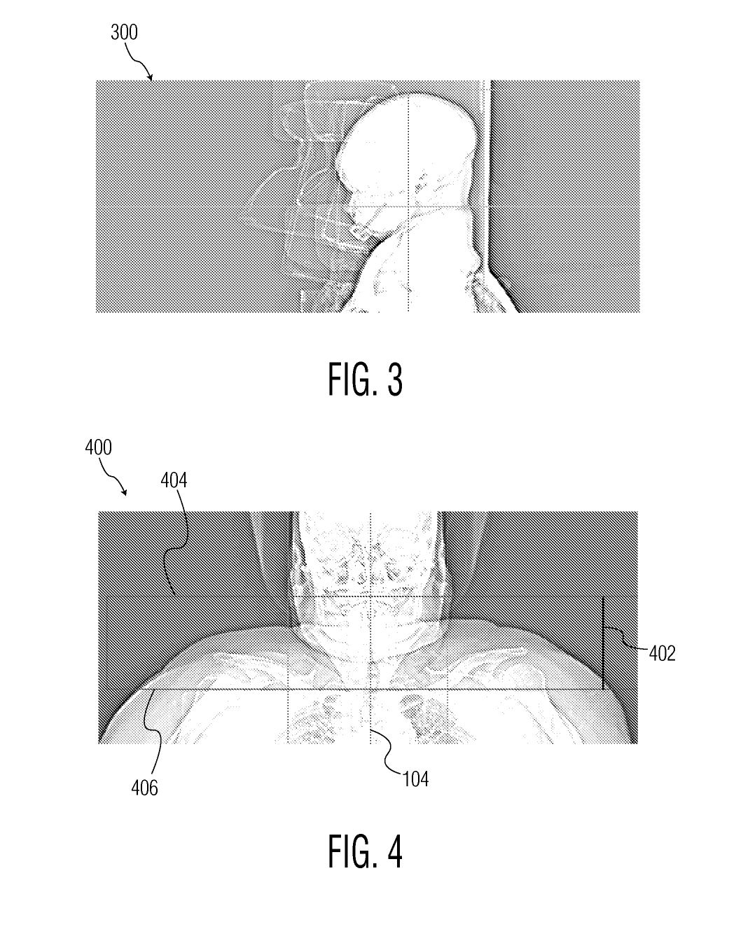

FIG. 3 is an exemplary side-view head section 300. The methods 800 and 1000 may be applied to the side-view head section 300 in a manner similar to their application to head section 200. Side-view head section may also have a head upper boundary 302 and a head lower boundary 304.

FIG. 4 is a neck section 400 of full-body input image 100. Neck section 400 is not a separate image, but is the portion of the full-body input image 100 below head section 200 or 300 (e.g., the section likely containing an image of a neck of the patient). Neck section 400 retains vertical symmetric axis 104. As will be discussed below with respect to methods 800 and 1100, neck section 400 may also have a neck rectangular region 402, a neck upper boundary 404 and a neck lower boundary 406.

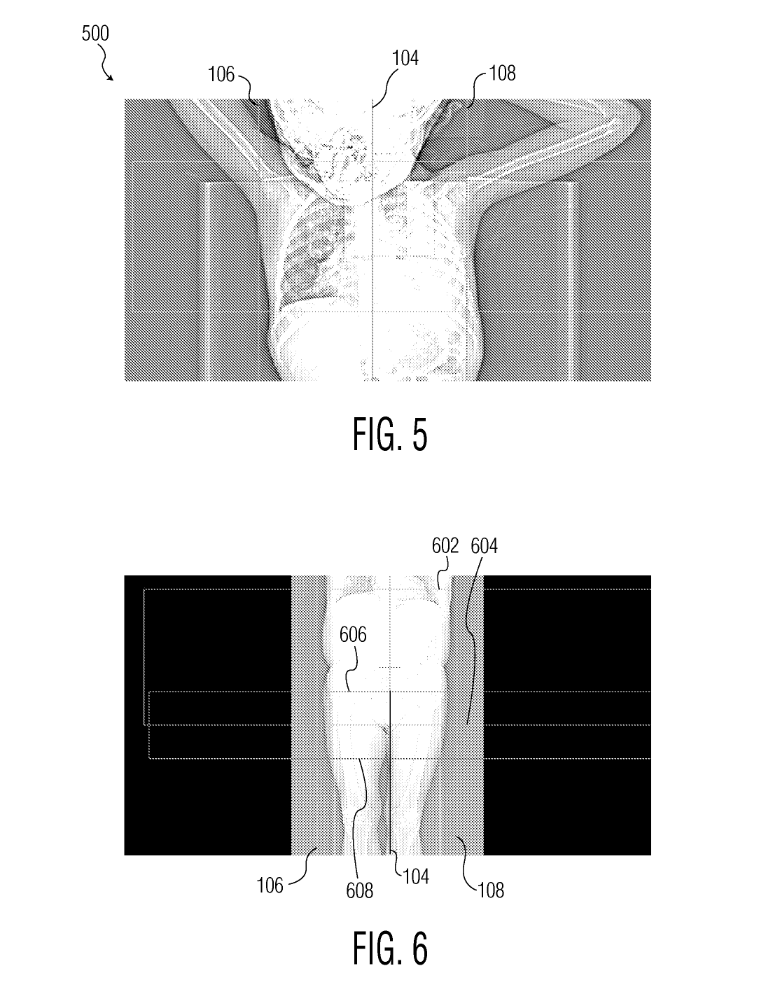

FIG. 5 is a thorax cage section 500 of full-body input image 100. Thorax cage section 500 is not a separate image, but is the portion of the full-body input image 100 below neck section 400 (e.g., the section likely containing an image of a thorax cage of the patient). Thorax cage section 500 retains vertical symmetric axis 104, left straight boundary 106, and right straight boundary 108. As will be discussed below with respect to methods 800 and 1200, thorax cage section 500 may also have a thorax cage upper boundary 502 and a thorax cage lower boundary 504.

FIG. 6 is an abdomen and pelvis section 600 of full-body input image 100. Abdomen and pelvis section 600 is not a separate image, but is the portion of the full-body input image 100 below thorax cage section 500 (e.g., the section likely containing an image of an abdomen and/or a pelvis of the patient). Abdomen and pelvis section 600 retains vertical symmetric axis 104, left straight boundary 106, and right straight boundary 108. As will be discussed below with respect to methods 800 and 1300, abdomen and pelvis section 600 may also have an abdomen upper boundary 602, an abdomen lower boundary 604, a pelvis upper boundary 606, and a pelvis lower boundary 608.

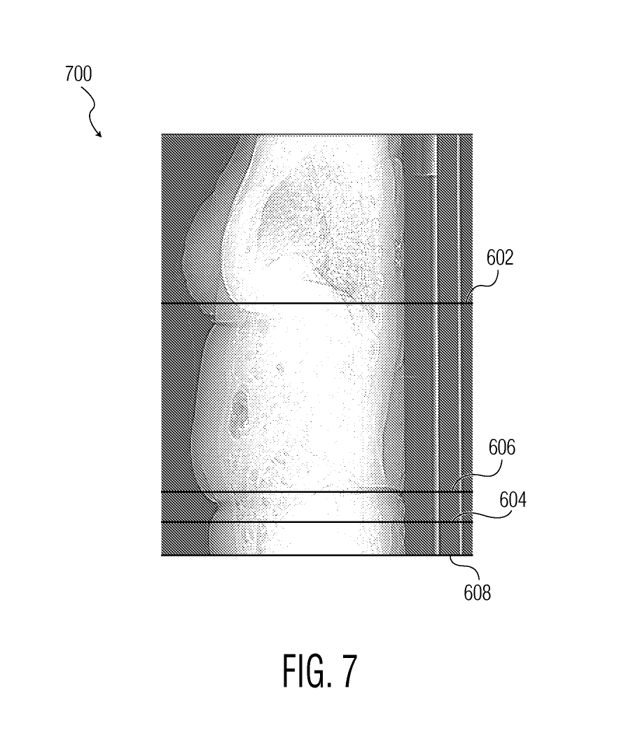

FIG. 7 is an exemplary side-view abdomen and pelvis section 700. Similar to abdomen and pelvis section 600, the methods 800 and 1200 may be applied to the side-view abdomen and pelvis section 700 which may also have an abdomen upper boundary 602, an abdomen lower boundary 604, a pelvis upper boundary 606, and a pelvis lower boundary 608.

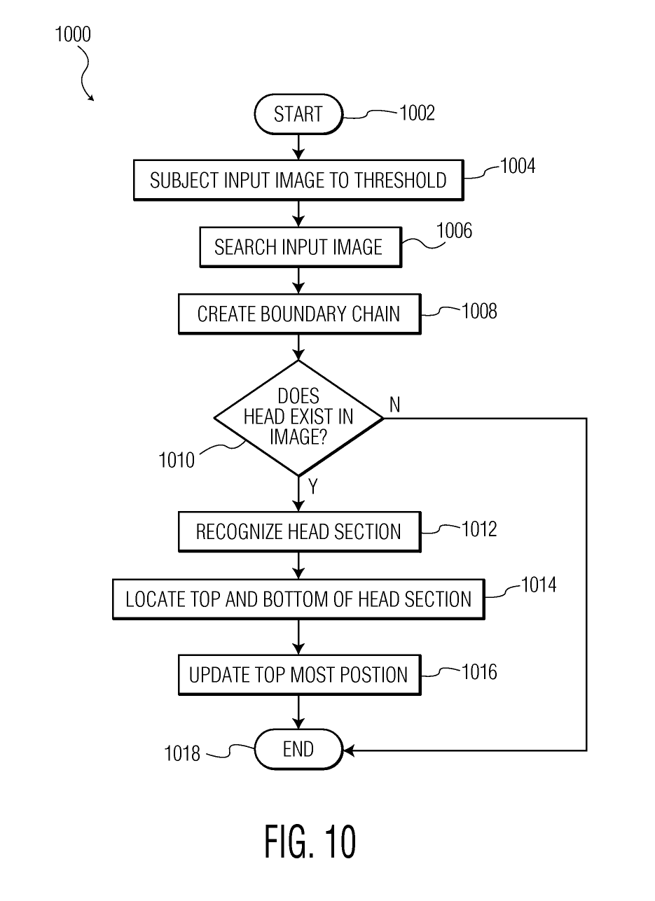

A method 800 of automatic body part identification and localization is depicted in FIG. 8. The body part identification and localization method 800 establishes a set of global reference properties which include a number of image geometries and intensity statistics to guide each identification and localization procedure to sequentially recognize and locate body parts in a number of methods 1000-1300, discussed in exemplary detail below with respect to FIGS. 10-13.

Though discussed with specificity in methods 800-1300, the techniques employed herein may be generalized for automatic body part identification and localization. For example, in a recognition module, an object may be recognized and at a localization module, a position (e.g., a starting and an ending position) of the object may be located.

As related to medical terminology in the specific examples herein, it is noted that the location and/or range of a body part is defined by medical imaging acquisition protocols which may differ from typical anatomical definitions. For example, the head section as used herein is analogous to the brain region in medical imaging acquisition protocols. The details of body part identification and localization algorithms are depicted in the following sections.

The method begins at step 802. In step 804, an input image (e.g., full-body input image 100) is received.

In step 806, a global reference frame is established in relation to the input image 100. In input images, the valid image size, image quality, intensity level, position and/or size of a body part vary significantly. A set of image properties which include a region of interest (e.g., ROI 102) in an input image, globe intensity statistics such as a pixel intensity histogram in a ROI and a min/max pixel intensity in a ROI, a vertical symmetric axis (e.g., vertical symmetric axis 104), left and right straight boundaries (e.g., left straight boundary 106 and right straight boundary 108), and the top most position (e.g., upper starting boundary 110) need to be estimated from the image to establish a global reference frame to guide the identification and localization of body parts. Further details are included below with respect to FIG. 9 and method 900.

In step 808, a first body part is identified and subsequently located. Based on current information such as the global reference frame and the indication that no previous body part identification had been made, the first body part may be identified as a primary or originating part. This may be a head section (as discussed with respect to method 1000 and FIGS. 2 and 10) or other body part that may be reliably and repeatably identified. Of course, any first body part may be identified and located using appropriate specific and/or generalized versions of the algorithms described herein. The first body part may then be located by assigning appropriate location information to the identified part in relation to the established global reference frame.

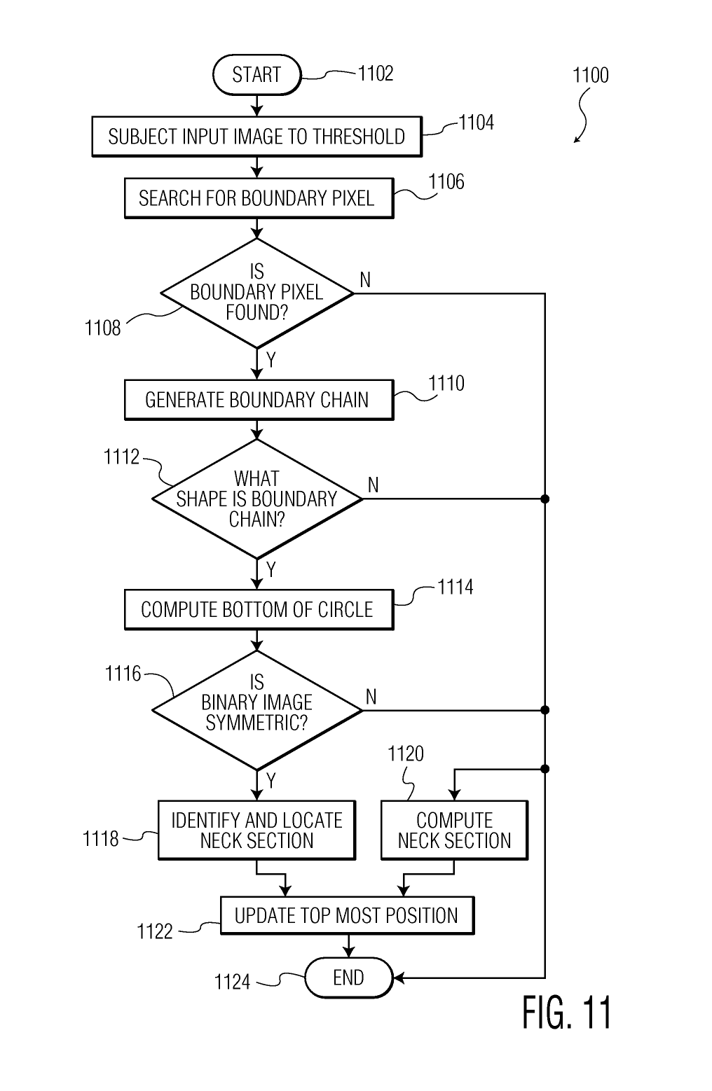

In step 810, a second body part is identified and located. The second body part may be any body part which may be identified based at least in part on its proximity, adjacency, and/or relation to the first body part identified and located in step 808. This may be a neck section as discussed with respect to FIGS. 3 and 11 and method 1100. Of course, any second body part may be identified and located using appropriate specific and/or generalized versions of the algorithms described herein. That is, after finding any appropriate first body part, a related (e.g., attached, adjacent, etc.) second body part may be identified and located. The second body part may be located at least in part on its relation to the first body part and/or the global reference frame. Thus, the identification and location of the second body part is contingent on the identification and location made in step 808.

In step 812, an N^{th} body part is identified and located. The N^{th} body part may be any body part which may be identified based at least in part on its proximity, adjacency, and/or relation to the first body part identified and located in step 808, the second body part identified in step 810, and/or any other body part identified in step 812. This may be a thorax cage section, an abdomen section, and/or a pelvis section as discussed with respect to FIGS. 4, 5, 12 and 13 and methods 1200 and 1300. Of course, any further (e.g., N^{th}) body part may be identified and located using appropriate specific and/or generalized versions of the algorithms described herein. That is, after finding any appropriate second body part in step 810, any number of subsequently related (e.g., attached, sequentially attached, unattached, etc.) N^{th} body parts may be identified and located. Thus, the N^{th} body part may be located at least in part on its relation to the first body part, the second body part, any other N^{th} body part and/or the global reference frame. Accordingly, the identification and location of the N^{th} body part is contingent on the identifications and locations made in steps 808, 810, and 812. The method ends at step 814.

FIG. 9 depicts a flowchart of a method 900 of establishing a global reference frame as in step 806 of method 800. The method begins at step 902.

In step 904, a region of interest (e.g., ROI 102) is computed. In some embodiments, the ROI 102 is computed as a rectangular region with one or more pixel intensity (e.g., brightness) distributions similar to those of the region at the center of the image, which may be heuristically defined as the rectangle region at the center of the image with 40% of image width and 40% of image height. A threshold-based segmentation with a predetermined threshold value (e.g., the 40% percentile of the intensity level at the center region, etc.) is applied to the input image 100 to produce a binary image. The binary image is then cleaned using morphological operations and connected component analysis. A rectangular bounding box of the cleaned binary image is computed as the ROI 102. After the ROI 102 is established, other reference properties (e.g., histogram and min/max intensity level, etc.) are computed within the ROI 102.

In step 906, vertical symmetric axis 104 is estimated. For example, based on a peak position of a smoothed x-profile of the input image 100, vertical symmetric axis 104 is determined (e.g., estimated) within the estimated ROI 102.

In step 908, after the vertical symmetric axis 104 is estimated, left straight boundary 106 and right straight boundary 108 are determined. In at least one embodiment, a search algorithm looks leftward and rightward from the vertical symmetric axis 104 to search for the very first position with an x-profile value less than a predetermined threshold (e.g., 10%) of the peak value in the left part and the right part of the smoothed x-profile to compute the left straight boundary 106 and the right straight boundary 108, respectively.

In step 910, the upper starting boundary 110 is determined. In at least one embodiment a profile jump detection algorithm, which detects the largest profile difference between a pair of consecutive neighboring sections (e.g., with length of 5) of y positions in a smoothed y-profile, is applied to detect the top most position of the image which is typically in the upper part of the input image. The smoothed x-profile and smoothed y-profile may be computed by applying a Gaussian filter (e.g., of size=7 and s.t.d.=2.0, etc.) a few (e.g., 2-5) times to the corresponding profiles, respectively. Of course, other methods of determining the upper boundary 110 may be used. The method ends at step 912.

FIG. 10 depicts an exemplary method 1000 of automatically identifying and locating a first body part, as in step 808 of method 800. For clarity, method 1000 describes one example of automatically identifying and locating a head section as in FIGS. 2 and 3. The method begins at step 1002.

In step 1004, input image 100 is subjected to a threshold to determine a binary image. That is, in at least one embodiment, a pixel with an intensity value larger than a pre-defined threshold value (e.g., between 1 and 0) is assigned. In the same or alternative embodiments, a predetermined intensity level (e.g., corresponding to a 40^{th} percentile of the intensity level in the ROI 102) is used. The resultant binary image is cleaned using morphological operations and connected component analysis similar to step 904 above.

In step 1006, input image 100 is searched to find whether the image depicts a full head (e.g., as in FIG. 2) or a side-view head (e.g., as in FIG. 3).

In step 1008, a boundary chain is created. Beginning with a current top most position (e.g., upper starting boundary 110) the vertical symmetric axis 104 is searched for a boundary pixel (e.g., point) as a starting point. A boundary pixel is a pixel on a segmented object that has neighboring background pixel(s). From the boundary pixel, leftward and rightward extensions generate a boundary chain.

In step 1010, it is determined it a head exists in the input image 100. That is, a head-like circular shape is searched for in the input image 100. The boundary chain is evaluated using an evaluation algorithm (e.g., a generalized Hough transform-based algorithm) to check whether the boundary chain forms a round shape centered about the vertical symmetric axis 104. If it does, then a head section is recognized (e.g., identified) and the method passes to step 1012. If not, the method ends at step 1018.

In step 1014, a top and bottom of the identified head section are located as the top and bottom of the circular shape that best match the round-shaped boundary chain. That is, head upper boundary 202 and a head lower boundary 204 are located.

In step 1016, the current top most position (e.g., upper starting boundary 110) is updated to a new current top most position at the bottom of the head section (e.g., head lower boundary 204 is also the new current top most position). The method ends at step 1018.

FIG. 11 depicts an exemplary method 1100 of automatically identifying and locating a second body part, as in step 810 of method 800. For clarity, method 1100 describes one example of automatically identifying and locating a neck section as in FIG. 4. The method begins at step 1102.

In step 1104, input image 100 is subjected to threshold using a predetermined intensity level (e.g., corresponding to a 40^{th} percentile of the intensity level in the ROI 102) and the resultant binary image is cleaned using morphological operations and connected component analysis similar to steps 1004 and 904 above.

In step 1106, a search is conducted to find a boundary pixel. In one embodiment, a search is conducted upward from the current top most position (e.g., head lower boundary 204) on the vertical symmetric axis 104. If no boundary pixel is found, a search is conducted downward from the current top most position (e.g., head lower boundary 204) on the vertical symmetric axis 104. If no boundary pixel is found, the method ends at step 1124.

A check is performed in step 1108 to determine if a boundary pixel is found during a search in step 1106. If a boundary pixel is found in any search, the found boundary pixel is treated as the starting pixel and a boundary chain is generated through leftward and rightward extension in step 1110, as above. If no boundary pixel is found, the method ends at step 1124.

In step 1112, the boundary chain is evaluated using an algorithm (e.g., a generalized Hough transform-based algorithm) to check whether the boundary chain forms a round or partial round shape (e.g., a circle) centered about the vertical symmetric axis 104. If such shapes are found, a neck section may be identified in steps 1114-1118. If not, the method ends at step 1124.

In step 1114, the bottom of the circle is computed to best match the round-shaped boundary chain. Then, in step 1116, the symmetry of the binary image is determined with respect to the vertical symmetric axis 104 inside a rectangular region 402 with a width of the diameter of the boundary chain and the height being twice the width.

If the binary image is symmetric, the method passes to step 1118. Here, a neck section is identified and located. In an exemplary embodiment, the neck section is identified with the neck upper boundary 404 located as bottom of the head (e.g., head lower boundary 204) minus 20% of the boundary chain circle diameter and the neck lower boundary 406 is located at the bottom of the boundary chain circle plus 200% of the circle diameter. Of course, other relationships to the head, head boundaries 202 and 204, and/or boundary chain circle may be used.

If the binary image is not symmetric, the method passes to step 1120 where the neck section is computed. Here, the y-profile of the neck section is computed. In at least one embodiment, the y-profile extends a distance of 200% of the boundary chain circle diameter from current top most position (e.g., head lower boundary 204) along the vertical symmetric axis 104. The largest profile difference between a pair of consecutive neighboring sections extending in the y-profile is estimated. If the estimated value is larger than a threshold (e.g., 10% of the mean profile), a neck section is identified. The position of the largest profile difference, increased by a percentage (e.g., 20%) of the circle diameter, is computed as the neck lower boundary 406. The neck upper boundary 404 is located at the bottom of the circle minus a percentage (e.g., 20%) of the circle diameter. Of course, other relationships to the head, head boundaries 202 and 204, and/or boundary chain circle may be used.

After either step 1118 or 1120, in step 1122, the top most position is updated to the bottom of the neck section. That is, the neck lower boundary 406 becomes the new current top most position. The method ends at step 1124.

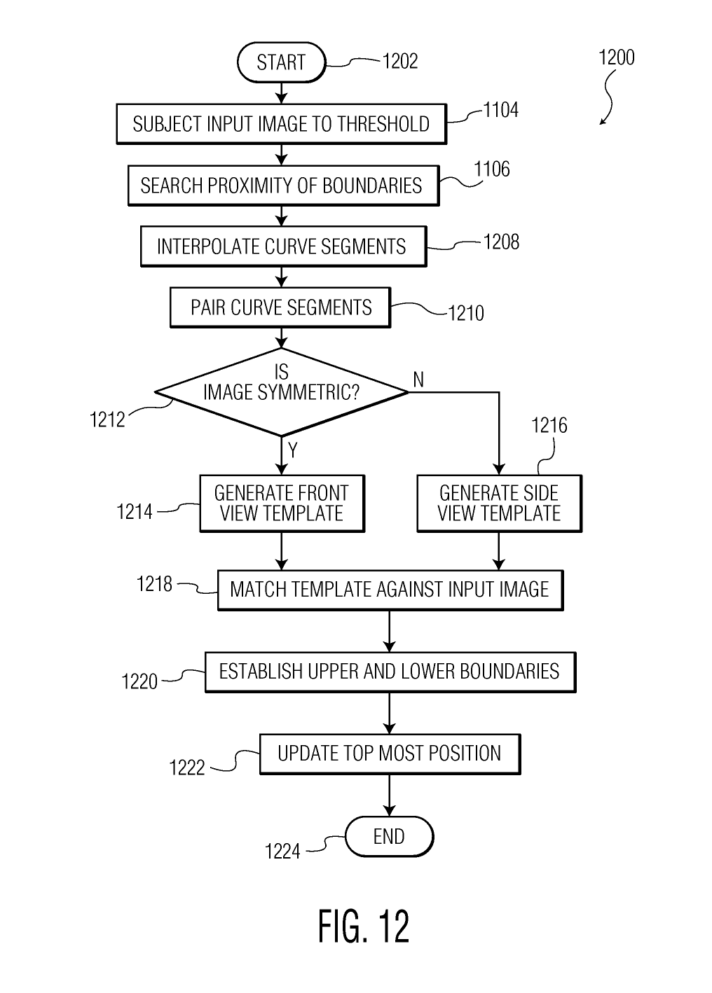

FIG. 12 depicts an exemplary method 1200 of automatically identifying and locating a third (N^{th}) body part, as in step 812 of method 800. For clarity, method 1200 describes one example of automatically identifying and locating a thorax cage section as in FIG. 5. The method begins at step 1202.

In step 1204, the input image 100 is subjected to threshold using a predetermined intensity value (e.g., corresponding to 75^{th} percentile intensity level in the ROI 102).

In step 1206, the proximity of the left straight boundary 106 and right straight boundary 108 is searched and all vertically formed and convex-like curve segments that are within a predetermined (e.g., within 20% of ROI 102) width away from the left straight boundary 106 or right straight boundary 108, respectively, are registered to form a left curve segment list and a right curve segment list. The searching region may be a rectangle at the current top most position (e.g., neck lower boundary 406) with a width approximately equal to a width of ROI 102 and a height of approximately 200% of the width.

In step 1208, an interpolation is performed between the low end of each curve segment in the curve segment lists and the vertical symmetric axis 104 with the upper section of an ellipse-like curve based on the tendency at the low end of the curve segment and the distance to the vertical symmetric axis 104 to form a curve segment starting from the vertical symmetric axis at top.

In step 1210, each interpolated curve segment is paired in one curve segment list with a curve segment in the other curve segment list that has similar distance to the vertical symmetric axis 104 and similar interpolation point on the vertical symmetric axis 104. If there is no corresponding interpolation point found, a symmetric mapping of the curve segment about the vertical symmetric axis 104 is generated as its symmetric partner.

In step 1212, a check is performed to determine, for each symmetric pair of curve segments, whether the image is symmetric with respect to the vertical symmetric axis 104. If it is symmetric, the method passes to step 1214 and a front view thorax cage-like template image is generated. If it is not symmetric, a side view thorax cage-like template image is generated at step 1216.

In step 1218, the generated template image is matched against the input image 100. If a normalized correlation coefficient is larger than a predetermined value (e.g., 0.4 for the front view and 0.3 for the side view), then a thorax cage section is identified. If the normalized correlation coefficient is not larger than the predetermined value, no thorax cage section is determined and the method end at step 1224.

Following thorax cage section identification in step 1218, the thorax cage upper boundary 502 is established at the interpolation point on the vertical symmetric axis 104 and the thorax cage lower boundary 504 is established as the lowest end of the paired curve segments in step 1220.

In step 1222, the top most position is updated as the thorax cage lower boundary 504. The method ends at step 1224.

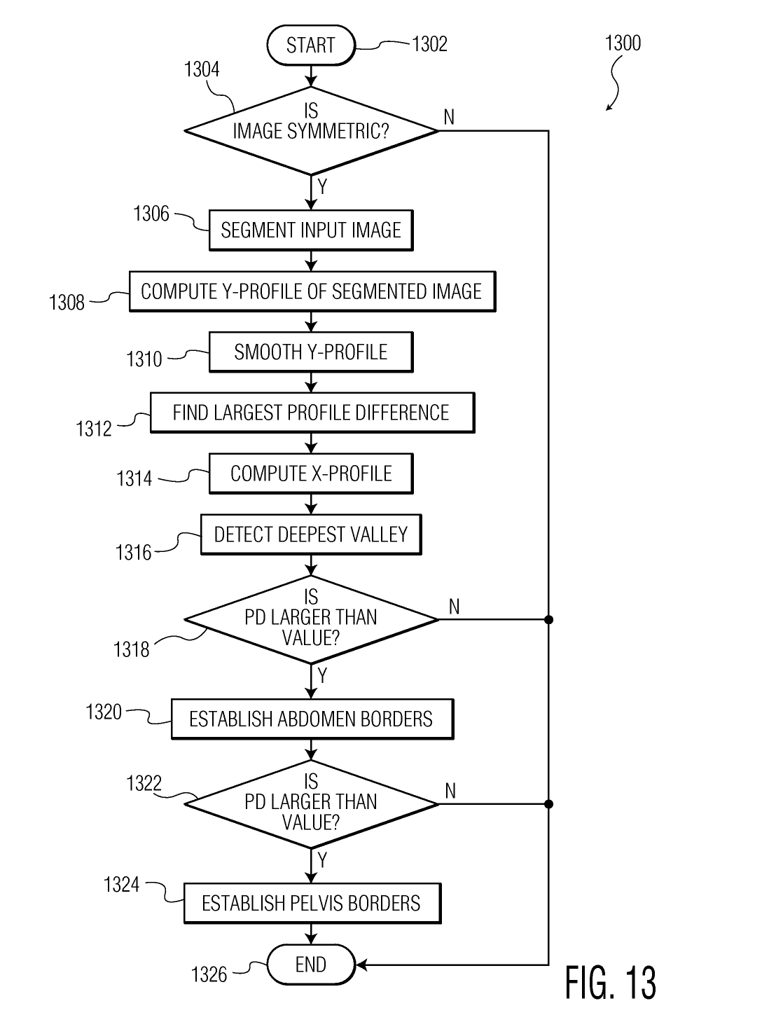

FIG. 13 depicts an exemplary method 1300 of automatically identifying and locating a fourth and/or fifth (N^{th}) body part, as in step 812 of method 800. For clarity, method 1300 describes an example of automatically identifying and locating an abdomen section and a pelvis section as in FIG. 6 or 7. There is no discriminating feature that can be easily computed to enable a reliable recognition of the abdomen section. Accordingly, the detection of the abdomen section may be combined with other body sections that can be easily identified. In general, if a valid abdomen section presents in input image 100, at the bottom end there exists at least a portion of the pelvis section. Therefore, it is beneficial to combine the detection of the abdomen section and the pelvis section into one procedure. The method begins at step 1302.

In step 1304, the input image 100 is checked to determine symmetry corresponding to the vertical symmetric axis 104 around the current top most position (e.g., thorax cage lower boundary 504). If input image 100 is not symmetric, the method ends at step 1326.

In step 1306, input image 100 is segmented from the top most position (e.g., thorax cage lower boundary 504) using a predetermined threshold (e.g., the 75^{th} percentile of the intensity level within two side regions next to a longitudinal (e.g., spinal) region) and the segmentation result is filtered using multi-pass median filter (e.g., of window size 3 pixels×3 pixels). A longitudinal (e.g., spinal) region is a region within 15% ROI width-span of the vertical symmetric axis.

In step 1308, the y-profile of the segmented image within the longitudinal region is computed. In step 1310, the y-profile is smoothed. For example, it may be smoothed using a Gaussian filter with size=5 and s.t.d.=2 a number (e.g., 5, etc.) times.

In stop 1312, the largest profile difference, PD, between a pair of consecutive neighboring sections in the smoothed y-profile is found. In at least one embodiment the section size is 5, though other sizes may be used.

In step 1314, the x-profile is computed at a local region starting from the y position corresponding to the largest PD, centered at the vertical symmetric axis 104 with both width and height being within a predetermined amount (e.g., within 50%) of the ROI 102 width. in step 1316, the deepest valley, PV, is detected near the center of the x-profile.

In step 1318, the PD is checked. If PD larger than 0.4 times the y-profile mean and PV larger than 0.2 times the x-profile mean then the abdomen section is identified and the method passes to step 1320. If not, the method ends at step 1326.

At step 1320, the abdomen boundaries are established. The abdomen upper boundary 602 is established as the top most position (e.g., thorax cage lower boundary 504) minus a percentage (e.g., 10%) of the ROI 102 width and the abdomen lower boundary 604 is established as the y-position of PV.

In step 1322, a check is performed to identify the pelvis section. If PD is larger than 0.4 times the y-protile mean, PV is larger than 0.2 times the x-profile mean, and the distance from PD to the bottom of the ROI is larger than a percentage (e.g., 15%) of the ROI 102 width, then a pelvis section is identified and the method passes to step 1324. Otherwise, the method ends at step 1326

In step 1324, the pelvis boundaries are established. In at least one embodiment, the pelvis upper boundary 606 is set as the y-position of PD minus a percentage (e.g., 30%) of the ROI 102 width and the pelvis lower boundary 608 is set as the y-position of PV plus a percentage (e.g., 15%) of the ROI 102 width.

The method ends at step 1326.

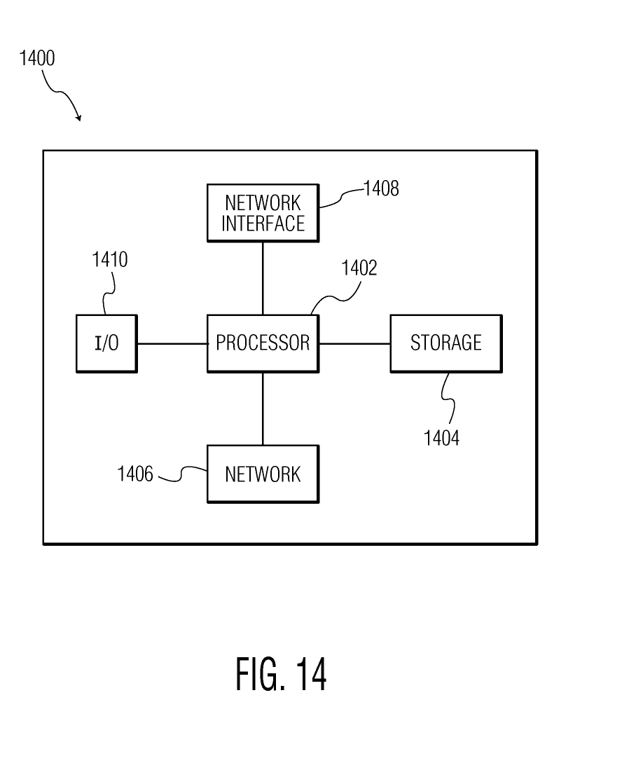

FIG. 14 is a detailed schematic drawing of a computer 1400 capable of carrying out the instructions embodied in methods 800-1300. Computer 1400 contains a processor 1402 which controls the overall operation of the computer 1400 by executing computer program instructions which define such operation. The computer program instructions may be stored in a storage device 1404 (e.g., magnetic disk, database, etc.) and loaded into memory 1406 when execution of the computer program instructions is desired. Thus, applications for performing the herein-described method steps, such as automatically identifying and locating body parts, may be defined by the computer program instructions stored in the memory 1406 and/or storage 1404 and controlled by the processor 1402 executing the computer program instructions. The computer 1400 also includes one or more network interfaces 1408 for communicating with other devices via a network. The computer 1400 also includes other input/output devices 1410 (e.g., display, keyboard, mouse, speakers, buttons, etc.) that enable user interaction with the computer 1400 such as transmitting, receiving, manipulating, or otherwise controlling an input image 100 as received in method 800. One skilled in the art will recognize that an implementation of an actual computer could contain other components as well, and that the computer of FIG. 14 is a high level representation of some of the components of such a computer for illustrative purposes.

Further, the computer 1400 may be implemented on, may be coupled to, and/or may include any components or devices that are typically used by, or used in connection with, a computer or computer system. Computer 1400 and/or processor 1402 may include one or more central processing units, read only memory (ROM) devices and/or random access memory (RAM) devices.

According to some embodiments of the present invention, instructions of a program (e.g., controller software) may be read into memory 1406, such as from a ROM device to a RAM device or from a LAN adapter to a RAM device. Execution of sequences of the instructions in the program may cause the computer 1400 to perform one or more of the method steps described herein. In alternative embodiments, hard-wired circuitry or integrated circuits may be used in place of, or in combination with, software instructions for implementation of the processes of the present invention. Thus, embodiments of the present invention are not limited to any specific combination of hardware, firmware, and/or software. The memory 1406 may store the software for the computer 1400, which may be adapted to execute the software program and thereby operate in accordance with the present invention and particularly in accordance with the methods described in detail above. However, it would be understood by one of ordinary skill in the art that the invention as described herein could be implemented in many different ways using a wide range of programming techniques as well as general purpose hardware sub-systems or dedicated controllers.

Such programs may be stored in a compressed, uncompiled and/or encrypted format. The programs furthermore may include program elements that may be generally useful, such as an operating system, a database management system and device drivers for allowing the controller to interface with computer peripheral devices, and other equipment/components. Appropriate general purpose program elements are known to those skilled in the art, and need not be described in detail herein.

The foregoing description discloses only particular embodiments of the invention; modifications of the above disclosed methods and apparatus which fall within the scope of the invention will be readily apparent to those of ordinary skill in the art. For instance, it will be understood that, though discussed primarily with specific body part identification algorithms in methods 1000-1300, other appropriate automatic detection, identification, and/or location may be used. Similarly, other components may perform the functions of methods 800-1300 even when not explicitly discussed.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

What is claimed is:

1. A method of automatic body part identification comprising:
establishing a reference frame in relation to an image;
identifying a first body section on the image; and
establishing a location of the first body section in relation to the reference frame.

2. The method of claim 1 further comprising:
identifying a second body section on the image based on the identified first body section; and
establishing a location of the second body section based at least in part on the location of the first body section.

3. The method of claim 1 wherein establishing a reference frame comprises:
computing a region of interest of the image;
estimating a vertical symmetrical axis of the image;
determining a left boundary substantially parallel to the vertical symmetrical axis;
determining a right boundary substantially parallel to the vertical symmetrical axis; and
determining an upper boundary at the top of the region of interest.

4. The method of claim 1 wherein identifying the first body section on the image comprises:
finding a first boundary point on the image;
creating a boundary chain of boundary points beginning at the first boundary point; and
evaluating the boundary chain with an evaluation algorithm to identify the first body section.

5. The method of claim 4 wherein establishing a location of the first body section in relation to the reference frame comprises:
determining an upper boundary of the first body section based on the evaluated boundary chain; and
determining a lower boundary of the first body section based on the evaluated boundary chain.

6. The method of claim 2 wherein identifying a second body section on the image based on the identified first body section comprises:
finding a first boundary point on the image based on a determined lower boundary of the first body section;
creating a boundary chain of boundary points beginning at the first boundary point; and
evaluating the boundary chain with an evaluation algorithm to identify the second body section.

7. The method of claim 6 wherein establishing a location of the second body section based at least in part on the location of the first body section comprises:
determining a symmetry of the image in relation to the reference frame;
determining an upper boundary of the first body section based on the determined symmetry of the image and the determined lower boundary of the first body section; and
determining a lower boundary of the first body section based at least in part on the evaluated boundary chain.

8. The method of claim 2 further comprising:
identifying a third body section on the image based on the identified second body section; and
establishing a location of the third body section based at least in part on the location of the second body section.

9. The method of claim 8 wherein identifying a third body section on the image based on the identified second body section and establishing a location of the third body section based at least in part on the location of the second body section comprises:
determining one or more curve segment lists comprising vertically formed and convex-like curve segments within a predetermined distance of a portion of the reference frame;
interpolating each curve segment between a lower end of the curve segment and a second portion of the reference frame;
pairing each interpolated curve segment with another curve segment that has a similar distance to the second portion of the reference frame;
determining if each pair of curve segments is symmetric with respect to the second portion of the reference frame;
generating a third body part template based on the determination;
matching the generated template to the image;
establishing a third body section upper boundary based at least in part on the interpolated curve segments and the second portion of the reference frame; and
establishing a third body section lower boundary based at least in part on the lower end of the paired curve segments.

10. The method of claim 8 further comprising:
identifying a fourth body section on the image based on the identified third body section; and
establishing a location of the fourth body section based at least in part on the location of the third body section
identifying a fifth body section on the image based on the identified fourth body section; and
establishing a location of the fifth body section based at least in part on the location of the fourth body section.

11. The method of claim 10 wherein identifying and establishing a location of the fourth and fifth body sections further comprises:
determining a symmetry of the image based on a portion of the reference frame;
segmenting the image from an established lower boundary of the third body section within a longitudinal region;
computing a y-profile of the segmented image;
smoothing the y-profile;
finding a largest profile difference between a pair of neighboring sections of the smoothed y-profile;
computing an x-profile based in part on the largest profile difference and a portion of the reference frame;
detecting the deepest valley of the x-profile;
identifying the fourth body section based at least in part on the x-profile and y-profile;
establishing an upper boundary of the fourth body section based at least in part on the established lower boundary of the third body section;
establishing a lower boundary of the fourth body section base at least in part on the y-position of the deepest valley of the x-profile.
identifying the fifth body section based at least in part on a profile difference and a portion of the reference frame;
establishing an upper boundary of the fifth body section based at least in part on the y-position of the profile difference; and
establishing an lower boundary of the fifth body section based at least in part on the y-position of the deepest valley.

12. The method of claim 1 wherein the first body section is a head section.

13. The method of claim 2 wherein the second body section is a neck section.

14. The method of claim 8 wherein the third body section is a thorax cage section.

15. The method of claim 10 wherein the fourth body section is an abdomen section and the fifth body section is a pelvis section.

16. A machine readable medium having program instructions stored thereon, the instructions capable of execution by a processor and defining the steps of:
establishing a reference frame in relation to an image;
identifying a first body section on the image; and
establishing a location of the first body section in relation to the reference frame.

17. The machine readable medium of claim 16, wherein the instructions further define the steps of:
identifying a second body section on the image based on the identified first body section; and
establishing a location of the second body section based at least in part on the location of the first body section.

18. The machine readable medium of claim 17, wherein the instructions further define the steps of:
identifying a third body section on the image based on the identified second body section; and
establishing a location of the third body section based at least in part on the location of the second body section.

19. The machine readable medium of claim 18, wherein the instructions further define the steps of:
identifying a fourth body section on the image based on the identified third body section; and
establishing a location of the fourth body section based at least in part on the location of the third body section.
identifying a fifth body section on the image based on the identified fourth body section; and
establishing a location of the fifth body section based at least in part on the location of the fourth body section.

20. An apparatus for automatic body part identification comprising:
means for establishing a reference frame in relation to an image;
means for identifying a first body section on the image; and
means for establishing a location of the first body section in relation to the reference frame.

21. The apparatus of claim 20 further comprising:
means for identifying a second body section on the image based on the identified first body section; and
means for establishing a location of the second body section based at least in part on the location of the first body section.

22. The apparatus of claim 21 further comprising:
means for identifying a third body section on the image based on the identified second body section; and
means for establishing a location of the third body section based at least in part on the location of the second body section.

23. The apparatus of claim 22 further comprising:
means for identifying a fourth body section on the image based on the identified third body section; and
means for establishing a location of the fourth body section based at least in part on the location of the third body section.
means for identifying a fifth body section on the image based on the identified fourth body section; and
means for establishing a location of the fifth body section based at least in part on the location of the fourth body section.
